(19) United States
(12) Patent Application Publication
(10) Pub. No.: US 2023/0203425 A1
(43) Pub. Date: Jun. 29, 2023

(54) METHOD FOR SEPARATING STROMAL VASCULAR FRACTION FROM ADIPOSE TISSUE USING NON-CONTACT ULTRASONIC DEVICE, AND NON-CONTACT ULTRASONIC DEVICE USED THEREIN

(71) Applicant: Min Ah YANG, Incheon (KR)

(72) Inventor: Min Ah YANG, Incheon (KR)

(21) Appl. No.: 18/076,288

(22) Filed: Dec. 6, 2022

(30) Foreign Application Priority Data
Dec. 24, 2021 (KR) 10-2021-0186942

(51) Int. Cl. C12M 1/00; C12N 5/0775

(52) U.S. Cl. CPC C12M 47/04; C12N 5/0667

(57) ABSTRACT

The present invention provides a method for separating a stromal vascular fraction (SVF) from adipose tissue by using a non-contact ultrasonic device comprising a power supply unit, an ultrasonic oscillation unit, and an ultrasonic vibration unit; and a non-contact ultrasonic device used therefor. Specifically, the present invention provides a method for separating a stromal vascular fraction (SVF) from adipose tissue, and a non-contact ultrasonic device used therefor, in which the stromal vascular fraction can be extracted from adipose tissue with high efficiency while minimizing cell contamination and destruction by using a non-enzymatic method and a non-contact ultrasonic device.

### CROSS-REFERENCE TO RELATED APPLICATIONS AND CLAIM OF PRIORITY

This application claims the benefit of Korean Patent Application No. 10-2021-0186942 filed Dec. 24, 2021, the content of which are fully incorporated herein by reference in its entirety.

### TECHNICAL FIELD

The present invention relates to a method for separating a stromal vascular fraction (SVF) from adipose tissue using a non-contact ultrasonic device.

Further, the present invention relates to a non-contact ultrasonic device having a non-contact ultrasonic method applied thereto and used in a method of separating a stromal vascular fraction (SVF) from adipose tissue.

### BACKGROUND ART

Adipose tissue-derived stromal vascular fraction (SVF) is a mixture of heterogeneous cells including preadipocytes, endothelial cells (EC), vascular endothelial progenitor cells (EPC), vascular smooth muscle cells (SMC), pericytes, parietal cells, macrophages, fibroblasts and adipose-derived stem/stromal cells (ASC), etc.

It has been reported that the stromal vascular fraction exhibits clinical effects in autoimmune and allergic pathologies such as Crohn's disease, graft-versus-host disease, multiple sclerosis and inflammatory bowel disease; clinical trials for the symptoms of acute myocardial infarction, lower extremity ischemia, incurable chronic wounds, radiation injury, and urinary incontinence, etc.; and several preliminary disease treatment models. In addition, the stromal vascular fraction is expected to prolong the survival of autologous fat transplantation and thus to be used as cosmetic and rehabilitation medicines.

Until now, when extracting the stromal vascular fraction from adipose tissue, a method of performing cell separation using an enzyme such as collagenase is mainly used. As an example, Korean Patent Laid-Open Publication No. 10-2018-0009435 discloses a method of separating the stromal vascular fraction from adipose tissue by treating washed adipose tissue with an enzyme formulation for tissue dissociation comprising one or more enzymes selected from the group consisting of collagenase, dispase and accutase.

However, in the case of the enzymatic method as disclosed in the above publication, it is not economical due to a large amount of time and cost required, and there is a problem of contamination by the enzyme or cell destruction due to the enzyme, and therefore, its use tends to be limited.

In order to solve this problem, in recent years, various attempts have been made to separate the stromal vascular fraction by using contact-type ultrasonic wave or centrifugation. However, in the case of the contact-type ultrasonic wave, there is a problem that the ultrasonic nozzle and the adipose tissue are in direct contact with each other, and thus contamination and cell destruction easily occur. In addition, in the case of the centrifugation, there is a problem that the number of cells in the stromal vascular fraction separated from the adipose tissue is remarkably small, and thus the efficiency is lowered.

Accordingly, the present applicant has developed a method for effectively separating a stromal vascular fraction from adipose tissue by applying non-contact ultrasonic wave, unlike conventional techniques such as an enzymatic method and a method using contact ultrasonic wave, and a non-contact ultrasonic device suitable therefor, thereby completing the present invention.

### PRIOR ART LITERATURE

### Patent Documents

(Patent Document 1) Korean Patent Laid-Open Publication No. 10-2018-0009435

### DISCLOSURE

### Technical Problem

A first object of the present invention is to provide a method for separating a stromal vascular fraction (SVF) in a non-enzymatic manner and by using a non-contact ultrasonic device, whereby the stromal vascular fraction can be extracted from adipose tissue with high efficiency while minimizing cell contamination or destruction.

A second object of the present invention is to provide a non-contact ultrasonic device which is used in the process of separating a stromal vascular fraction from adipose tissue so as to minimize cell contamination or destruction while separating the stromal vascular fractions with high efficiency.

The objects of the present invention are not limited to the technical problem as described above, and another technical problem may be derived from the following description.

### Technical Solution

In order to achieve the above first object, the present invention provides a method for separating a stromal vascular fraction (SVF) using a non-contact ultrasonic device, wherein the stromal vascular fraction is separated from adipose tissue by using a non-contact ultrasonic device comprising a power supply unit, an ultrasonic oscillation unit, and an ultrasonic vibration unit, the method including: an injection step of injecting the adipose tissue into an inner space of a first tubular container; an ultrasonic irradiation step of mounting the first tubular container having the adipose tissue injected therein on the ultrasonic vibration unit of the non-contact ultrasonic device and irradiating ultrasonic waves to separate the adipose tissue; a centrifugation step of mounting the first tubular container having the adipose tissue injected therein on a centrifuge and performing centrifugation to separate the adipose tissue into oil, collagen, and the stromal vascular fraction; a washing step of injecting the stromal vascular fraction and a washing solution into an inner space of a second tubular container and performing centrifugation; and an extraction step of extracting the stromal vascular fraction washed according to the washing step.

In the ultrasonic irradiation step, the ultrasonic waves having a frequency of 30 to 40 kHz may be irradiated.

In the ultrasonic irradiation step, the ultrasonic waves may be irradiated for 1 to 10 minutes.

The ultrasonic oscillation unit may include a horn.

The horn of the ultrasonic oscillation unit and/or the ultrasonic vibration unit may be rotatable.

The horn of the ultrasonic oscillation unit and/or the ultrasonic vibration unit may be rotatable at an angle of 120° to 240°.

The horn of the ultrasonic oscillation unit and/or the ultrasonic vibration unit may be rotatable by the maximum angle for a predetermined time, wherein the predetermined time may be 1 second to 10 seconds.

The horn of the ultrasonic oscillation unit and/or the ultrasonic vibration unit may be stopped for a preset time after being rotated by the maximum angle, wherein the preset time may be 1 second to 10 seconds.

The horn of the ultrasonic oscillation unit and/or the ultrasonic vibration unit may irradiate the ultrasonic waves for the preset time.

An emulsification phenomenon may be alleviated by the rotation of the horn.

In the centrifugation step, the centrifugation may be performed for 1 to 5 minutes at a rotation speed of 800 to 1,000 rpm.

In the washing step, the centrifugation may be performed for 3 to 5 minutes at a rotation speed of 800 to 1,000 rpm.

The separation method may be a non-enzymatic method.

In order to achieve the above second object, the present invention provides a non-contact ultrasonic device for separating a stromal vascular fraction (SVF), wherein the stromal vascular fraction is separated from adipose tissue according to the above method, the non-contact ultrasonic device comprising: a power supply unit; an ultrasonic oscillation unit for generating ultrasonic waves by using an electric energy supplied from the power supply unit; and an ultrasonic vibration unit formed in the shape of a quadrangular column in contact with the ultrasonic oscillation unit, wherein a plurality of fixing grooves are formed on the upper surface of the column such that a tubular container accommodating the adipose tissue is inserted from the upper surface to the lower surface and fixed thereto.

The fixing grooves may be at least two or more.

### Advantageous Effects

According to the method provided by the present invention, it is possible to separate the stromal vascular fraction from adipose tissue with high efficiency while minimizing cell contamination and destruction by using a non-enzymatic method and a non-contact ultrasonic device.

In addition, the non-contact ultrasonic device provided herein enables efficient separation of the stromal vascular fraction from adipose tissue while minimizing cell contamination and destruction.

### DESCRIPTION OF DRAWINGS

FIG. 1 is a flowchart showing a sequence of a method for separating a stromal vascular fraction from adipose tissue according to the present invention.

FIG. 2 is a view schematically illustrating a part of the method for separating a stromal vascular fraction from adipose tissue.

FIG. 3 is a perspective view of a non-contact ultrasonic device according to an embodiment of the present invention.

FIG. 4 is a perspective view of a non-contact ultrasonic device according to another embodiment of the present invention.

FIG. 5 is a plan view of an ultrasonic oscillation unit and an ultrasonic vibration unit in the non-contact ultrasonic device shown in FIG. 3.

FIG. 6 is a side view of an ultrasonic oscillation unit and an ultrasonic vibration unit in the non-contact ultrasonic device shown in FIG. 3.

FIG. 7 is a view illustrating a state in which a tubular container is mounted to the non-contact ultrasonic device shown in FIG. 3.

FIG. 8 is a result confirming that an emulsification phenomenon is alleviated by the method of separating a stromal vascular fraction from adipose tissue according to the present invention.

BEST MODES OF THE INVENTION

Hereinafter, with reference to the accompanying drawings, embodiments of the present invention will be described in detail so as to be easily implemented by one of ordinary skill in the art to which the present invention pertains. However, the present invention may be embodied in a variety of forms and is not be limited to the embodiments described herein. In order to clearly describe the present invention in the drawing, parts irrelevant to the description are omitted from the drawings.

The terms or words used in the specification and claims of the present application are not to be construed as being limited to their ordinary or dictionary meanings, but should be interpreted as meanings and concepts consistent with the technical spirit of the present invention, based on the principle that the inventor may adequately define the concepts of terms to best describe his invention.

Throughout the specification of the present application, when it is described as “including” a certain element, it means that other elements may be further included, rather than excluded, unless otherwise stated.

Throughout the specification of the present application, the phrase “A and/or B” means A, B, or A and B.

Hereinafter, the present invention will be specifically described, but the present invention is not limited thereto.

The present invention provides a method for separating a stromal vascular fraction (SVF) from adipose tissue by using a non-contact ultrasonic device, and a non-contact ultrasonic device used therefor.

According to an embodiment of the present invention, there is provided a method for separating a stromal vascular fraction (SVF) using a non-contact ultrasonic device, wherein the stromal vascular fraction is separated from adipose tissue by using a non-contact ultrasonic device comprising a power supply unit, an ultrasonic oscillation unit, and an ultrasonic vibration unit, the method including: an injection step of injecting the adipose tissue into an inner space of a first tubular container; an ultrasonic irradiation step of mounting the first tubular container having the adipose tissue injected therein on the ultrasonic vibration unit of the non-contact ultrasonic device and irradiating ultrasonic waves to separate the adipose tissue; a centrifugation step of mounting the first tubular container having the adipose tissue injected therein on a centrifuge and performing centrifugation to separate the adipose tissue into oil, collagen, and the stromal vascular fraction; a washing step of injecting the stromal vascular fraction and a washing solution into an inner space of a second tubular container and performing centrifugation; and an extraction step of extracting the stromal vascular fraction washed according to the washing step.

According to the separation method of the present embodiment, the stromal vascular fraction can be separated from adipose tissue with high efficiency while minimizing cell contamination and destruction by using a non-enzymatic method and a non-contact ultrasonic device.

Hereinafter, each step of the separation method and a non-contact ultrasonic device used therein will be described in detail with reference to the drawings.

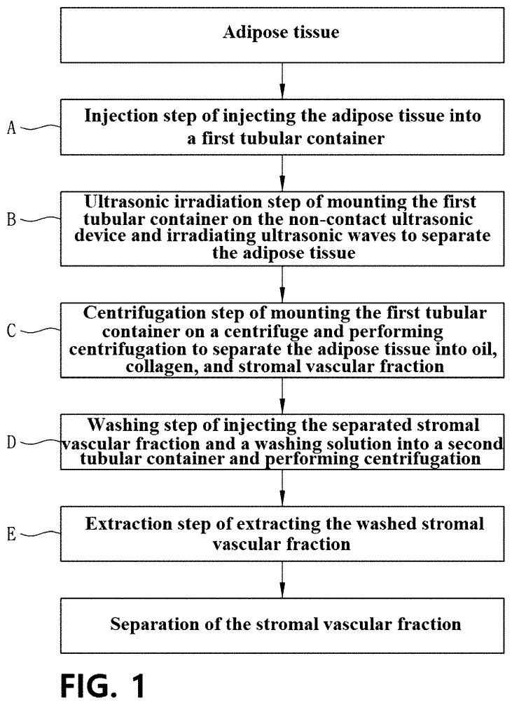

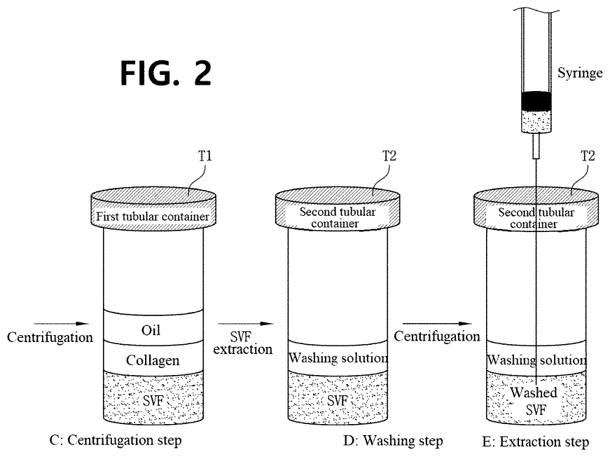

Specifically, FIG. 1 is a flowchart showing a sequence of the method for separating a stromal vascular fraction from adipose tissue according to the present embodiment, and FIG. 2 is a view schematically showing this. As shown in FIGS. 1 and 2, the separation method of the present embodiment may include an injection step, an ultrasonic irradiation step, a centrifugation step, a washing step, and an extraction step.

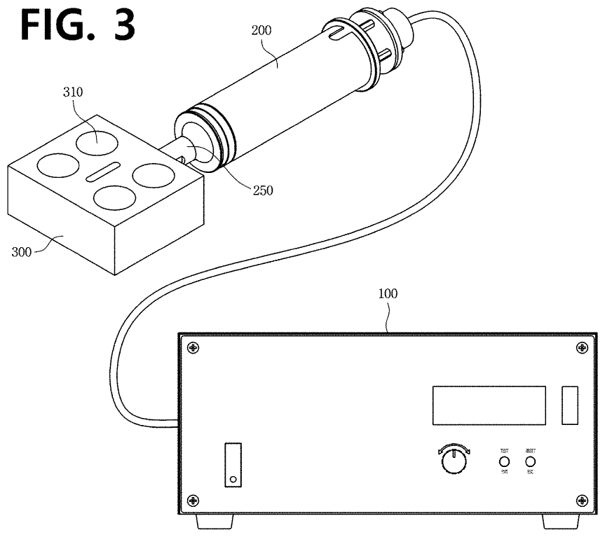

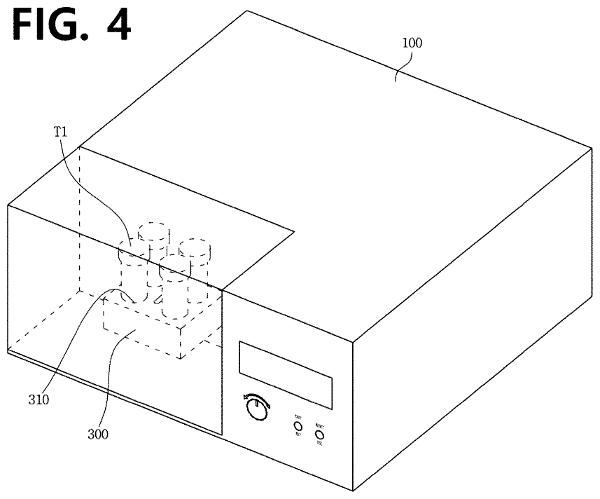

Further, FIG. 3 is a perspective view of a non-contact ultrasonic device according to an embodiment of the present invention, and FIG. 4 is a perspective view of a non-contact ultrasonic device according to another embodiment of the present invention.

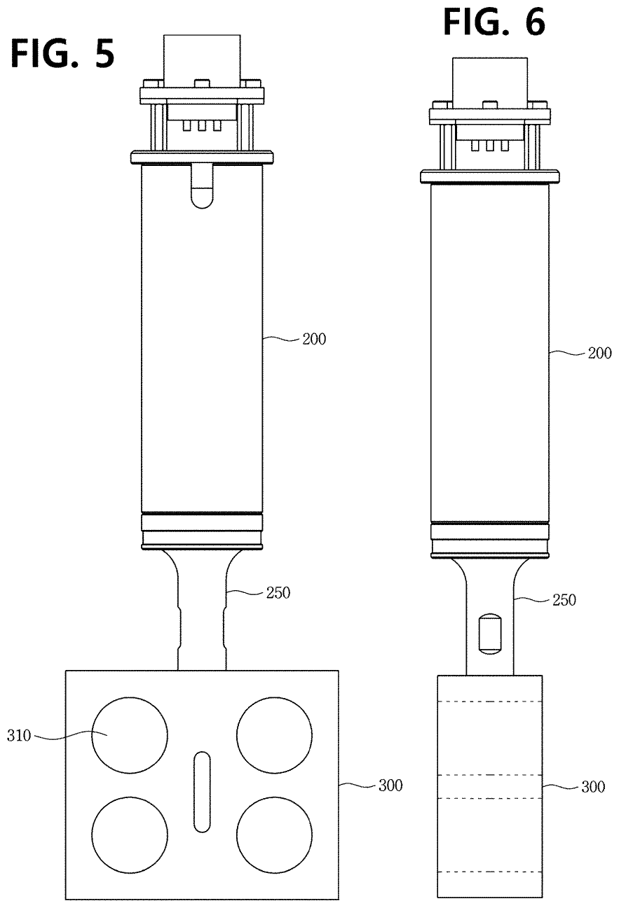

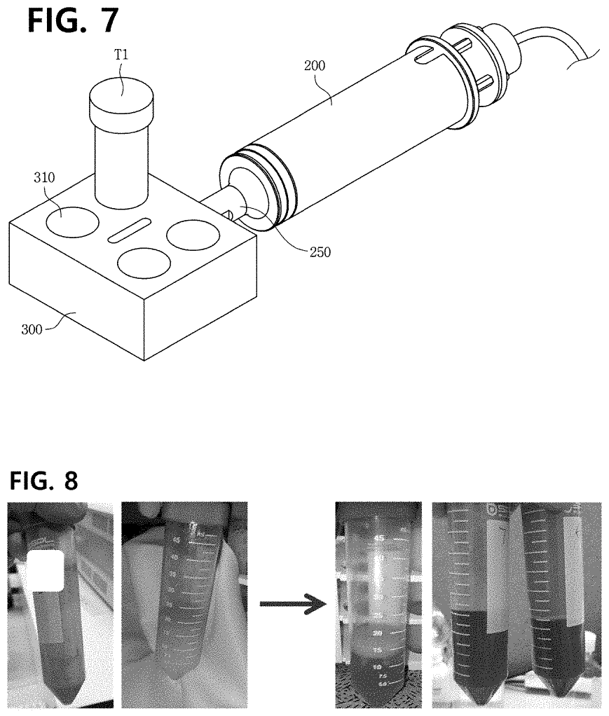

In addition, FIG. 5 is a plan view of the non-contact ultrasonic device shown in FIG. 3; FIG. 6 is a side view of the non-contact ultrasonic device shown in FIG. 3; and FIG. 7 is a view illustrating a state in which a first tubular container is mounted to the non-contact ultrasonic device shown in FIG. 3.

(A) Injection Step:

In the injection step of the present embodiment, the adipose tissue may be injected into an inner space of a first tubular container T1. Specifically, as shown in FIG. 1, in the injection step of this embodiment, the adipose tissue may be injected into the inner space of the first tubular container T1 through an inlet of the first tubular container T1.

(B) Ultrasonic Irradiation Step:

In the ultrasonic irradiation step of the present embodiment, the first tubular container T1 into which the adipose tissue is injected in the injection step may be mounted on the non-contact ultrasonic device 1, and the adipose tissue may be separated by irradiating ultrasonic waves.

Here, as shown in FIGS. 3 to 6, the non-contact ultrasonic device 1 includes a power supply unit 100; an ultrasonic oscillation unit 200 for generating ultrasonic waves by using an electric energy supplied from the power supply unit 100; and an ultrasonic vibration unit 300 formed in the shape of a quadrangular column in contact with the ultrasonic oscillation unit 200, wherein a plurality of fixing grooves 310 are formed on the upper surface of the column such that a tubular container T1 accommodating the adipose tissue is inserted from the upper surface to the lower surface and fixed thereto.

In particular, in the non-contact ultrasonic device 1 of the present embodiment, the power supply unit 100, the ultrasonic oscillation unit 200, and the ultrasonic vibration unit 300 may be formed separately from each other as shown in FIG. 3; or the ultrasonic oscillation unit 200 and the ultrasonic vibration unit 300 may be formed to be positioned in the inner space of the power supply unit 100 as shown in FIG. 4.

As shown in FIG. 3, the power supply unit 100 of the present embodiment supplies electric energy to the non-contact ultrasonic device 1 of the present embodiment, and the form and configuration thereof are not particularly limited. Here, the power supply unit 100 of the present embodiment may include an electric energy supply switch, and a control box capable of adjusting the frequency and irradiation time of ultrasonic waves, etc., but is not limited thereto.

As shown in FIGS. 3 to 6, the ultrasonic oscillation unit 200 of the present embodiment generates ultrasonic waves by using an electric energy supplied from the power supply unit 100, and the form and configuration thereof are not particularly limited. For example, the ultrasonic oscillation 200 of the present embodiment may be formed in a form in which a converter and a booster are sequentially connected, and may not be exposed to the outside by being surrounded by a housing.

Additionally, the ultrasonic oscillation unit 200 according to the present embodiment may include a horn 250, but may not include the horn 250. If the ultrasonic oscillation unit 200 of the present embodiment does not include the horn 250, the ultrasonic vibration unit 300 of the present embodiment formed to be in contact with the ultrasonic oscillation unit 200 may serve as a conventional horn.

As shown in FIGS. 3 to 6, the ultrasonic vibration unit 300 of the present embodiment is formed in the shape of a quadrangular column in contact with the ultrasonic oscillation unit 200, wherein a plurality of fixing grooves 310 are formed on the upper surface of the column such that a tubular container T1 accommodating the adipose tissue is inserted from the upper surface to the lower surface and fixed thereto. As shown in FIGS. 3 and 4, the fixing grooves 310 may be formed in at least two or more, preferably at least four or more, but is not limited thereto.

Here, as shown in FIG. 3, the ultrasonic oscillation unit 200 may be located on the side of the ultrasonic vibration unit 300 of the present embodiment; or the ultrasonic oscillation unit 200 may be positioned on the lower surface of the ultrasonic vibrating unit 300 of the present embodiment.

As shown in FIGS. 3 to 6, in the present embodiment, the contamination and destruction of cells can be minimized by irradiating ultrasonic waves in a non-contact manner, in which the adipose tissue is injected into the first tubular container T1 without direct contact of the ultrasonic probe with the adipose tissue, and the first tubular container T1 is inserted and fixed to the ultrasonic vibration unit 300.

In addition, when the ultrasonic oscillation unit 200 of the present embodiment includes the horn, the horn of the ultrasonic oscillation unit 200 and the ultrasonic vibration unit 300 may be rotated together with the ultrasonic oscillation unit 200 as a reference axis. Further, when the ultrasonic oscillation unit 200 of the present embodiment does not include the horn, the ultrasonic vibration unit 300 may be rotated with the ultrasonic oscillation unit 200 as a reference axis. In particular, as described above, when the horn of the ultrasonic oscillation unit 200 and/or the ultrasonic vibration unit 300 are rotated, the adipose tissue accommodated in the first tubular container T1 is uniformly mixed and subjected to ultrasonic waves during the ultrasonic irradiation process.

The horn of the ultrasonic oscillation unit 200 and/or the ultrasonic vibration unit 300 may be rotatable at an angle of 120° to 240°, preferably 150° to 210°, more preferably 180°. When rotated at an angle smaller than the above rotation angle, the effects of emulsification or cell separation may be lowered; and when rotated at an angle greater than the above rotation angle, oil (fatty oil) may flow out of the adipose tissue. The horn of the ultrasonic oscillation unit 200 and/or the ultrasonic vibration unit 300 may be rotated by the maximum angle for a predetermined time, and then be stopped for a preset time. The predetermined time may be set as an any time between 1 second and 10 seconds. In one embodiment, the predetermined time may be from 1 second to 5 seconds, preferably from 1 second to 3 seconds. The preset time may be determined as any time between 1 second and 10 seconds. In one embodiment, the predetermined time may be from 1 second to 5 seconds, preferably from 1 second to 3 seconds. The ultrasonic waves are not irradiated while the horn of the ultrasonic oscillation unit 200 and/or the ultrasonic vibration unit 300 is rotated by the maximum angle, but may be irradiated while they are stopped. The emulsification phenomenon may be alleviated by the rotation of the horn of the ultrasonic oscillation unit 200 and/or the ultrasonic vibration unit 300.

Specifically, in the ultrasonic irradiation step of the present embodiment, the first tubular container T1 into which the adipose tissue is injected in the injection step is inserted and fixed in the fixing groove 310 of the ultrasonic vibration unit 300; the ultrasonic waves are generated from the ultrasonic oscillation 200 by supplying power to the power supply unit 100; and the generated ultrasonic waves reach the first tubular container T1 fixed to the ultrasonic vibration unit 300, thereby separating the adipose tissue accommodated in the first tubular container T1.

The frequency of the ultrasonic wave may be from 30 to 40 kHz, specifically from 32 to 38 kHz, more specifically from 33 to 37 kHz. In particular, according to this embodiment, when ultrasonic waves having a frequency of 33 to 37 kHz are irradiated in the ultrasonic irradiation step of the present embodiment, the stromal vascular fraction can be separated from the adipose tissue with high efficiency while using the non-enzymatic method and the non-contact ultrasonic device 1.

If the frequency of the ultrasonic wave is less than 30 kHz, the separation efficiency of the stromal vascular fraction from the adipose tissue may be reduced. On the other hand, when the frequency of the ultrasonic wave is greater than 40 kHz, cell damage may occur, and thus the separation efficiency of the stromal vascular fraction may be lowered.

The frequency of the ultrasonic wave is 30 to 40 kHz, and may be irradiated for 1 to 10 minutes, specifically for 3 to 9 minutes, more specifically for 5 to 8 minutes.

In particular, according to this embodiment, when ultrasonic waves having a frequency of 30 to 40 kHz are irradiated for 5 to 8 minutes in the ultrasonic irradiation step of the present embodiment, the stromal vascular fraction can be separated from the adipose tissue with high efficiency while using the non-enzymatic method and the non-contact ultrasonic device 1.

If the ultrasonic irradiation time is less than 1 minute, the separation efficiency of the stromal vascular fraction from the adipose tissue may be reduced. On the other hand, when the ultrasonic irradiation time exceeds 10 minutes, cell damage may occur, and thus the separation efficiency of the stromal vascular fraction may be lowered.

(C) Centrifugation Step:

As shown in FIG. 2, in the centrifugation step of the present embodiment, the first tubular container T1 having the adipose tissue injected therein and subjected to the ultrasonic irradiation step may be mounted on a centrifuge and subjected to centrifugation to be separated into oil, collagen and the stromal vascular fraction. In particular, according to the present embodiment, the ultrasonic irradiation step is performed using the non-contact ultrasonic device 1, and then the centrifugation is performed using the centrifugal separator, whereby the stromal vascular fraction can be more effectively separated from the adipose tissue than when only the ultrasonic waves are irradiated or when only the centrifugation is performed.

Here, the centrifugation may be performed at a rotation speed of 800 to 1,000 rpm, specifically 900 to 1,000 rpm. In particular, according to the present embodiment, when the centrifugation is performed at a rotation speed of 900 to 1,000 rpm, the stromal vascular fraction can be separated from the adipose tissue with high efficiency while using the non-enzymatic method and the non-contact ultrasonic device 1.

If the rotation speed of the centrifugation is less than 800 rpm, the separation efficiency of the stromal vascular fraction from the adipose tissue may be reduced. On the other hand, when the rotation speed of the centrifugation exceeds 1,000 rpm, cell damage may occur due to excessive rotation and vibration, and thus the separation efficiency of the stromal vascular fraction may be lowered.

The centrifugation may be performed at a rotation speed of 800 to 1,000 rpm for 1 to 5 minutes, specifically 2 to 4 minutes. In particular, according to the present embodiment, when the centrifugation is performed for 2 to 4 minutes, the stromal vascular fraction can be separated from the adipose tissue with high efficiency while using the non-enzymatic method and the non-contact ultrasonic device 1.

If the centrifugation is performed for less than about 1 minute, the separation efficiency of the stromal vascular fraction from the adipose tissue may be reduced. On the other hand, if the centrifugation is performed for more than 5 minutes, cell damage may occur due to excessive rotation and vibration, and thus the separation efficiency of the stromal vascular fraction may be lowered.

(D) Washing Step:

As shown in FIG. 2, in the washing step of the present embodiment, the stromal vascular fraction is separately taken from the oil, collagen and stromal vascular fractions separated in the centrifugation step, and injected into the inner space of the second tubular vessel T2; and a washing solution for washing is also injected therein; and then, the centrifugation may be performed to effect washing.

The washing solution may include at least one selected from the group consisting of normal saline (NS), Hank's Balanced Salt Solution (HBSS), and phosphate-buffered saline (PBS). Specifically, the washing solution may be 1 to 10% phosphate-buffered saline (PBS) solution, but is not limited thereto.

In the washing step of the present embodiment, after the stromal vascular fraction and the washing solution are injected into the second tubular vessel T2, centrifugation is performed, wherein the centrifugation may be performed for 3 to 5 minutes at a rotation speed of 800 to 1,000 rpm, specifically 850 to 950 rpm. In particular, according to the present embodiment, when the centrifugation is performed at 850 to 950 rpm in the washing step of the present embodiment, the stromal vascular fraction can be separated from the adipose tissue with high efficiency while using the non-enzymatic method and the non-contact ultrasonic device 1.

If the rotation speed of the centrifugation is less than 800 rpm, the separation efficiency of the stromal vascular fraction from the adipose tissue may be reduced. On the other hand, when the rotation speed of the centrifugation exceeds 1,000 rpm, cell damage may occur due to excessive rotation and vibration, and thus the separation efficiency of the stromal vascular fraction may be lowered.

(E) Extraction Step:

As shown in FIG. 2, in the extraction step of the present embodiment, the washed stromal vascular fraction may be extracted from the separated washing solution and the washed stromal vascular fraction by using a known tool. Specifically, in the extraction step of the present embodiment, only the washed stromal vascular fraction can be separately extracted using a syringe.

In the separation method of the present embodiment, a known pre-treatment or post-treatment step for increasing the separation or extraction efficiency of the stromal vascular fraction from the adipose tissue may be further performed.

In addition, the non-contact ultrasonic device 1 of the present embodiment may further include a known means for increasing the separation or extraction efficiency of the stromal vascular fraction from the adipose tissue.

Hereinafter, with regard to the method for separating a stromal vascular fraction (SVF) from adipose tissue by using a non-contact ultrasonic device, and the non-contact ultrasonic device used therefor, they will be specifically described by way of Examples, Comparative Examples, and Experimental Examples. These examples are only for illustrating the present invention and thus should not be construed as limiting the scope of the present invention thereto.

### EXAMPLES

### Example 1: Separation of Stromal Vascular Fraction from Adipose Tissue

(A) Adipose tissue was injected into a first tubular container.

(B) As shown in FIG. 7, the first tubular container into which the adipose tissue was injected was inserted into and fixed to a fixing groove of an ultrasonic vibration unit of a non-contact ultrasonic device.

Then, power was supplied to a power supply unit of the non-contact ultrasonic device to generate ultrasonic waves, wherein the ultrasonic waves were irradiated at a frequency of 33 to 37 kHz for about 5 minutes. The ultrasonic waves were not irradiated for 2 seconds during which the horn of the ultrasonic oscillation unit and the ultrasonic vibration unit were rotated, but were irradiated for 2 seconds when they were stopped, and such process was repeated.

(C) After the ultrasonic waves were irradiated, the first tubular container was mounted on a centrifuge, and then centrifuged for 3 minutes at a rotation speed of about 900 to 1,000 rpm, whereby as shown in FIG. 2, the layers were separated into oil, collagen and stromal vascular fraction.

(D) Only the stromal vascular fraction accommodated in the first tubular container was extracted with a syringe and then injected into a new second tubular container. Then, a 5% PBS solution (washing solution) was injected into the second tubular container and washed by centrifugation for 3 to 5 minutes at a rotation speed of 850 to 950 rpm.

(E) As shown in FIG. 2, among the washing solution and the washed stromal vascular fraction separated by the centrifugation, the stromal vascular fraction was extracted from the second tubular container by using a syringe.

### COMPARATIVE EXAMPLE

### Comparative Example 1

According to an enzymatic method, a stromal vascular fraction was separated from adipose tissue.

Specifically, 1 mL of adipose tissue and 1 mL of collagenase were mixed and reacted at about 37° C. for 40 minutes, followed by centrifugation (1,500 rpm, 5 minutes), and the precipitated stromal vascular fraction was extracted. This fraction was washed by mixing it with a washing solution, centrifuged (1,500 rpm, 5 min), and then the washed stromal vascular fraction was extracted.

### Comparative Example 2

Except for omitting the above step (B), it was carried out in the same manner as in Example 1.

### Comparative Example 3

Except for omitting the above step (C), it was carried out in the same manner as in Example 1.

### Comparative Examples 4 to 7

Except that in the above step (B), the ultrasonic waves were irradiated under the conditions shown in Table 1, they were carried out in the same manner as in Example 1.

TABLE 1

| | Comparative | Comparative | Comparative | Comparative | Comparative |
|---|---|---|---|---|---|
| | Example 4 | Example 5 | Example 6 | Example 7 | Example 8 |
| Ultrasonic | 20~25 kHz | 45~50 kHz | 33~37 kHz | 33~37 kHz | 40~45 kHz |
| frequency | | | | | |
| Irradiation | 5 minutes | 5 minutes | 30 seconds | 15 minutes | 5 minutes |
| time | | | | | |

### Comparative Examples 9 to 12

Except that in the above step (C), the centrifugation was performed under the conditions shown in Table 2, they were carried out in the same manner as in Example 1.

TABLE 2

| | Comparative | Comparative | Comparative | Comparative |
|---|---|---|---|---|
| | Example 9 | Example 10 | Example 11 | Example 12 |
| Revolutions | 600~700 | 1,100~1,200 | 900~1,000 | 900~1,000 |
| per minute | | | | |
| (rpm) | | | | |
| Time | 3 minutes | 3 minutes | 30 seconds | 10 minutes |

### Comparative Example 13

Except that in the above step (B), the ultrasonic waves were continuously irradiated for 3 to 5 minutes without stopping, it was carried out in the same manner as in Example 1.

### EXPERIMENTAL EXAMPLE

Experimental Example 1: Evaluation of the number of stem cells in the stromal vascular fraction

The number of stem cells contained in the stromal vascular fraction separated from 1 ml of adipose tissue according to Example 1 was measured using an automatic cell measuring instrument NC-200 (stem cell number/fat tissue 1 mL), and the results are shown in Table 3.

In addition, the number of stem cells contained in the stromal vascular fraction separated from 1 ml of adipose tissue according to Comparative Examples 1 to 13 was measured, and the results are shown in Table 3.

TABLE 3

| | Number of cells |
|---|---|
| Example 1 | 65,800 |
| Comparative | 42,000 |
| Example 1 | |
| Comparative | 36,000 |
| Example 2 | |
| Comparative | 15,600 |
| Example 3 | |
| Comparative | 20,000 |
| Example 4 | |
| Comparative | 34,500 |
| Example 5 | |
| Comparative | 21,100 |
| Example 6 | |
| Comparative | 37,000 |
| Example 7 | |
| Comparative | 15,000 |
| Example 8 | |
| Comparative | 30,200 |
| Example 9 | |
| Comparative | 35,000 |
| Example 10 | |
| Comparative | 28,000 |
| Example 11 | |
| Comparative | 34,500 |
| Example 12 | |
| Comparative | 0 |
| Example 13 | |

Referring to Table 3, it can be seen that according to the present example, the separation efficiency of stem cells in the stromal vascular fraction from adipose tissue is remarkably excellent, which means that the stromal vascular fraction is separated from fat cells with high efficiency.

Specifically, it can be seen that Example 1 according to the present invention has better separation efficiency of the stromal vascular fraction than Comparative Example 1 which is an enzymatic method.

In addition, when comparing Example 1 with Comparative Examples 2 to 3, it can be seen that the separation efficiency of the stromal vascular fraction is improved only when both non-contact ultrasonication and centrifugation are performed.

Further, upon comparison of Example 1 with Comparative Example 8, it can be seen that when irradiating the ultrasonic waves with a frequency of 40 to 45 kHz in step (B), the cell yield is remarkably low.

Further, upon comparison of Example 1 with Comparative Examples 4 to 12, it can be seen that when the ultrasonic waves having a frequency of 30 to 40 kHz is irradiated for 1 to 10 minutes in step (B), and when the centrifugation is performed for 1 to 5 minutes at a rotation speed of 800 to 1,000 rpm in step (C), the separation efficiency of the stromal vascular fraction is significantly improved.

Experimental Example 2: Evaluation of Separation Performance of the Stromal Vascular Fraction

The stromal vascular fractions were separated from 1 g of adipose tissue according to Example 1 and Comparative Example 1, respectively, and the total number of cells in the separated stromal vascular fraction was measured. The results are shown in Table 4.

In addition, the survival rate was measured using an automatic cell counting equipment (Luna Stem from Logos, or Countess II automated cell counter from Life technologies) for the stromal vascular fraction, and the results are shown in Table 4.

TABLE 4

| | Number of cells in the | |
|---|---|---|
| | stromal vascular fraction | Survival rate |
| Example 1 | 7.45 × 10⁵ cells/g | 92.0% |
| Comparative | 4.00 × 10⁵ cells/g | 75.3% |
| Example 1 | | |

Referring to Table 4, it can be seen that in Example 1 using the non-contact ultrasonic method according to the present invention, the separation efficiency of the stromal vascular fraction and also the survival rate are excellent.

In contrast, it can be seen that in the case of Comparative Example 1 using the enzymatic method unlike the present invention, the separation efficiency of the stromal vascular fraction and also the cell survival rate are lower than in Example 1.

That is, according to the present invention, it is possible to effectively separate the stromal vascular fraction from the adipose tissue by performing the ultrasonication and centrifugation under specific conditions while using the non-enzymatic method and the non-contact ultrasonic method.

### Experimental Example 3: Evaluation of the Number of Stem Cells According to the Ultrasonic Irradiation Method

The stromal vascular fractions were separated from 1 g of adipose tissue according to Example 1 and Comparative Example 13, respectively, and the total number of cells in the separated stromal vascular fraction was measured. As a result, when the ultrasonic waves were continuously irradiated for 3 to 5 minutes without stopping (Comparative Example 13), it was confirmed that the adipose tissue was burned by frictional heat, and the cell survival rate was very low (number of cells 0, survival rate 0%).

It is to be understood that the description of the present invention as described above is for illustrative purposes only, and can be easily modified into other specific embodiments by those of ordinary skill in the art to which the present invention pertains without changing the technical spirit or essential features of the present invention. Therefore, it should be understood that the embodiments described above are illustrative in all respects and not restrictive. For example, each element described as a single form may be implemented dispersedly, and likewise elements described as dispersed may be embodied in a combined form.

The scope of the present invention is indicated by the following claims rather than the above detailed description, and all changes or modifications derived from the meaning and scope of the claims and their equivalents should be construed as belonging to the scope of the present invention.

### DESCRIPTION OF REFERENCE NUMERALS AND SYMBOLS

A: Injection step
B: Ultrasonic irradiation step
C: Centrifugation step
D: Washing step
E: Extraction step
T1: First tubular container
T2: Second tubular container
1: Non-contact ultrasonic device
100: power supply unit
200: Ultrasonic oscillation unit
250: Horn
300: Ultrasonic vibration unit
310: Fixing groove

1. A method for separating a stromal vascular fraction using a non-contact ultrasonic device, wherein the stromal vascular fraction is separated from adipose tissue by using a non-contact ultrasonic device comprising a power supply unit, an ultrasonic oscillation unit, and an ultrasonic vibration unit, the method including:
an injection step of injecting the adipose tissue into an inner space of a first tubular container;
an ultrasonic irradiation step of mounting the first tubular container having the adipose tissue injected therein on the ultrasonic vibration unit of the non-contact ultrasonic device and irradiating ultrasonic waves to separate the adipose tissue;
a centrifugation step of mounting the first tubular container having the adipose tissue injected therein on a centrifuge and performing centrifugation to separate the adipose tissue into oil, collagen, and the stromal vascular fraction;
a washing step of injecting the stromal vascular fraction and a washing solution into an inner space of a second tubular container and performing centrifugation; and
an extraction step of extracting the stromal vascular fraction washed according to the washing step.

2. The method for separating a stromal vascular fraction using a non-contact ultrasonic device according to claim 1, wherein in the ultrasonic irradiation step, the ultrasonic waves having a frequency of 30 to 40 kHz are irradiated.

3. The method for separating a stromal vascular fraction using a non-contact ultrasonic device according to claim 1, wherein in the ultrasonic irradiation step, the ultrasonic waves are irradiated for 1 to 10 minutes.

4. The method for separating a stromal vascular fraction using a non-contact ultrasonic device according to claim 1, wherein the ultrasonic oscillation unit includes a horn.

5. The method for separating a stromal vascular fraction using a non-contact ultrasonic device according to claim 4, wherein the horn of the ultrasonic oscillation unit and/or the ultrasonic vibration unit are rotatable.

6. The method for separating a stromal vascular fraction using a non-contact ultrasonic device according to claim 5, wherein the horn of the ultrasonic oscillation unit and/or the ultrasonic vibration unit are rotatable at an angle of 120° to 240°.

7. The method for separating a stromal vascular fraction using a non-contact ultrasonic device according to claim 4, wherein the horn of the ultrasonic oscillation unit and/or the ultrasonic vibration unit are rotatable by the maximum angle for a predetermined time.

8. The method for separating a stromal vascular fraction using a non-contact ultrasonic device according to claim 7, wherein the horn of the ultrasonic oscillation unit and/or the ultrasonic vibration unit are stopped for a preset time after being rotated by the maximum angle.

9. The method for separating a stromal vascular fraction using a non-contact ultrasonic device according to claim 8, wherein the horn of the ultrasonic oscillation unit and/or the ultrasonic vibration unit irradiate the ultrasonic waves for the preset time.

10. The method for separating a stromal vascular fraction using a non-contact ultrasonic device according to claim 1, wherein in the centrifugation step, the centrifugation is performed for 1 to 5 minutes at a rotation speed of 800 to 1,000 rpm.

11. The method for separating a stromal vascular fraction using a non-contact ultrasonic device according to claim 1, wherein in the washing step, the centrifugation is performed for 3 to 5 minutes at a rotation speed of 800 to 1,000 rpm.

12. The method for separating a stromal vascular fraction using a non-contact ultrasonic device according to claim 1, wherein the separation method is a non-enzymatic method.

13. A non-contact ultrasonic device for separating a stromal vascular fraction (SVF), wherein the stromal vascular fraction is separated from adipose tissue according to the method of claim 1, the non-contact ultrasonic device comprising:
a power supply unit;
an ultrasonic oscillation unit for generating ultrasonic waves by using an electric energy supplied from the power supply unit; and
an ultrasonic vibration unit formed in the shape of a quadrangular column in contact with the ultrasonic oscillation unit, wherein a plurality of fixing grooves are formed on the upper surface of the column such that a tubular container accommodating the adipose tissue is inserted from the upper surface to the lower surface and fixed thereto.

14. The non-contact ultrasonic device for separating a stromal vascular fraction according to claim 13, wherein the fixing grooves are at least two or more.
